(19) United States
(12) Patent Application Publication
(10) Pub. No.: US 2015/0366677 A1
(43) Pub. Date: Dec. 24, 2015

(54) IMPACT RESISTANT MEDICAL INSTRUMENTS, IMPLANTS AND METHODS

(71) Applicant: SMITH & NEPHEW, INC., Memphis, TN (US)

(72) Inventors: Alec Paul Porzel, Collierville, TN (US); Thomas William Lux, Collierville, TN (US); Robert H. Dyer, Bartlett, TN (US)

(73) Assignee: SMITH & NEPHEW, INC., Memphis, TN (US)

(21) Appl. No.: 14/767,788

(22) PCT Filed: Feb. 11, 2014
(86) PCT No.: PCT/US2014/015770

§ 371 Date: Aug. 13, 2015

Related U.S. Application Data
(60) Provisional application No. 61/764,387, filed on Feb. 13, 2013.

(51) Int. Cl. A61F 2/46; A61B 17/92; B29C 43/00; A61F 2/38

(52) U.S. Cl. CPC A61F 2/4684; A61F 2/3859; A61F 2/461; A61B 17/92; B29C 43/006; A61F 2240/007; A61F 2002/3863; A61F 2002/4623; A61F 2002/4681; B29L 2031/753

(57) ABSTRACT

Embodiments of the invention include surgical instruments, implants, and methods. Surgical instruments or implants may be manufactured from a mixture of a polymer and a filler material. In some embodiments, the polymer is a medical grade polymer and the filler is a reinforcing material that increases the rigidity of the mixture when cured. The polymer may include one or both of a USP Class VI approved base resin and an ISO 10993-1 approved base resin in some embodiments.

### CROSS REFERENCE TO RELATED APPLICATIONS

The present application claims the benefit of U.S. Provisional Application Ser. No. 61/764,387 filed Feb. 13, 2013, the contents of which are incorporated herein by reference in their entirety.

### FIELD OF THE INVENTION

The present invention relates generally to the field of medical instruments and methods, and more particularly relates to surgical instruments, implants, and methods, where the surgical instruments or implants have been manufactured from materials including at least a polymer and a filler material.

### BACKGROUND

Many surgical instruments and implants require or benefit from particular levels of impact resistance and material rigidity. For example, a surgical instrument or implant may require a certain level of material rigidity to adequately transfer force through the surgical instrument or implant. An implant may be required to approximate the rigidity of a physiological structure that it is designed to replace. At the same time, it is often useful or even necessary to impact a surgical instrument or implant during a surgical procedure. However, impact resistance and material rigidity characteristics may have an inverse relationship. In many materials, as rigidity goes up so does brittleness, and a brittle material has a lower impact resistance and lower material toughness.

Many polymer materials are known to have good impact resistance and material toughness, but may lack adequate rigidity. Therefore, it is known in the art to mix filler materials with polymer materials to improve the rigidity of the mixture when cured. However, such known polymer materials may not be approved for use in surgical procedures as either an instrument or an implant. Consequently, current solutions may involve the use of more expensive material blends or may involve overmolding plastic or rubber materials around metal structures. All of these solutions increase the expense of instruments and implants and may increase expense to a level where it is not possible to provide, for example, disposable surgical instruments.

Improved instruments, implants, and methods may provide for adequately impact resistant and rigid instruments and implants that are constructed from materials that have been evaluated and approved for use as surgical devices.

### SUMMARY

An embodiment of the invention is a femoral trial that includes at least a trial instrument made generally in the shape of a femoral component implant that is configured to receive impact for the purpose of seating the trial instrument on a distal end of a femur of a patient. The trial instrument may be manufactured at least in part from a medical grade polymer and a filler material capable of increasing the rigidity of the trial instrument when mixed with the medical grade polymer.

Another embodiment of the invention is a surgical instrument configured to be sterilized and used to manipulate the tissue of a patient. The surgical instrument embodiment includes a surgical instrument body including a force receiving zone that is configured to receive impact to position the surgical instrument within the patient or to manipulate the tissue of the patient. The surgical instrument may be manufactured at least in part from a medical grade polymer and a filler material capable of increasing the rigidity of the surgical instrument when mixed with the medical grade polymer.

Still another embodiment of the invention is a method of constructing a thermoplastic composite material that has the ability to be formed into a surgical instrument that is configured to receive an impact force. This method embodiment includes at least obtaining a medical grade polymer, obtaining a filler material capable of increasing the rigidity of the thermoplastic composite material when consolidated with the medical grade polymer, consolidating the medical grade polymer with the filler to create a mixture, and pressing the mixture into a mold constructed in the shape of the surgical instrument configured to receive an impact force.

Yet another embodiment of the invention is a method of supplying a material acceptable for use as a surgical device. This method may include at least providing a polymer cleared for use during a surgical procedure for the benefit of a patient, providing a filler material configured to increase the rigidity of the polymer when consolidated with the polymer, consolidating the polymer with the filler to create a mixture, evaluating the mixture for suitability, and making the material available for use as part of a surgical device.

### BRIEF DESCRIPTION OF THE DRAWINGS

FIG. 1 is a perspective view of an embodiment of a femoral trial being placed onto a distal end of a femur.

FIG. 2 is a perspective view of the femoral trial illustrated in FIG. 1.

### DETAILED DESCRIPTION

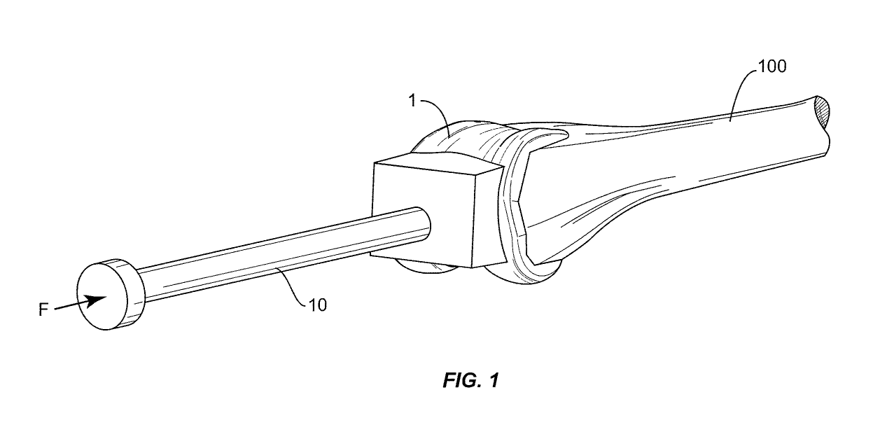

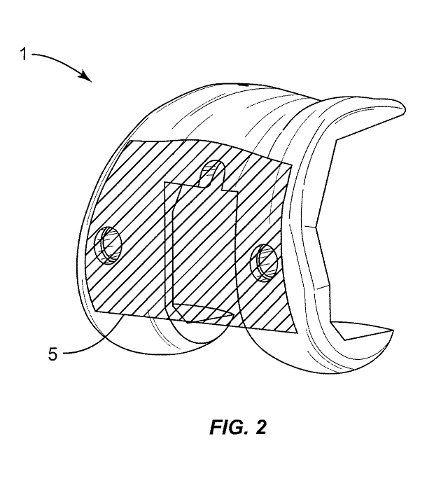

Instruments and implants manufactured at least in part from a polymer and a filler material are contemplated. In an example embodiment, a surgical instrument is a femoral trial 1. As shown in FIG. 1, the femoral trial 1 is being placed onto a distal end of a femur 100. The femoral trial 1 is a ream-through femoral trial, which means that the femoral trial 1 is configured to receive a reamer through its central portion such that a patient's femur 100 may be accessed while the femoral trial 1 is in place on the femur 100. An impactor 10 is shown receiving an impact force F to drive the femoral trial 1 onto the femur 100. This impact force F may be applied by, without limitation, a mallet, a hammer, a slap hammer, an automated impaction tool, or any other tool or device capable of delivering a force to drive the impactor 10 toward the femoral trial 1. In FIG. 2, a force receiving or impaction zone 5 is shown on the femoral trial 1 that corresponds to the area that would receive force from the impactor 10, as illustrated in FIG. 1. The femoral trial 1 must be rigid enough to retain its shape when impacted, but impact resistant enough to not fracture when impacted. Material of the femoral trial 1 in and near the impaction zone 5 is particularly susceptible to fracture when impacted.

The femoral trial 1 is a trial instrument made generally in the shape of a femoral component implant of a total knee arthroplasty device. In other embodiments, an instrument or implant of the invention may be any component, part, or sub-part of a medical instrument or implant. The femoral trial 1 is configured to receive impact, such as the impactor 10, to seat the femoral trial 1 on a distal end of a femur 100 of a patient. The illustrated trial instrument, femoral trial 1, is manufactured at least in part from a polymer and a filler material capable of increasing the rigidity of the trial instrument when mixed with the polymer. The femoral trial 1 is an instrument for use in an orthopedic procedure, but in other embodiments, an instrument or implant of the invention may be any other medical instrument or implant and may be for any medical procedure.

An instrument of some embodiments of the invention, such as the femoral trial 1, may be a disposable device. For example, an instrument set may include two or more trials of various sizes so that the trials can be placed in contact with a patient's tissue to judge the appropriate size of a device to ultimately be implanted. Such instruments may be packaged in sterile packaging or may be delivered for surgery non-sterile and sterilized by surgical staff members. It may be an advantage in some circumstances to provide instruments that can be used intraoperatively and then disposed of, thereby reducing the costs associated with additional cleaning, sterilization, and possibly packaging. This option may also reduce the potential for contaminant or disease transmission by improving the quality and repeatability of sterilization procedures by accomplish these tasks in a more quality controlled environment. Sterilization may include steam sterilization with an autoclave, or sterilization by one or more of the following: ethylene oxide, vaporized hydrogen peroxide, plasma vaporized hydrogen peroxide, electron beam, or gamma radiation. Any other effective sterilization technique may also be used.

A polymer for use in various embodiments of the invention may include any feasible polymer resin or material. Some polymers for use in various embodiments of the invention may particularly be medical grade polymers. Medical grade polymers include properties that make the polymers useful in medical procedures. Example useful properties, among others, may include non-irritation of a patient's tissue, less susceptibility to leaching, and an absence of an immune response in the presence of the polymer. Polymers of some embodiments may be polymers with relatively high temperature resistance characteristics.

Some polymers of embodiments of the invention may be polycarbonates, which are a particular group of thermoplastic polymers. Polycarbonates are polymers containing carbonate groups. Many polycarbonates are derived from rigid monomers, and may have a balance of useful features including temperature resistance, impact resistance, chemical stability, and optical properties. Polycarbonates may have the additional benefit of being less expensive than engineering plastics, although offering many of the same benefits. Embodiments of the invention may particularly include Poly (bisphenol-A-carbonate) polycarbonates. One example medical grade polymer that is a Poly (bisphenol-A-carbonate) is a product trade named LEXAN (a product of SABIC Innovative Plastics) and has the product identification HPH4404-7H6D057T. This material may be referred to as LEXAN HPH4404.

LEXAN HPH4404, as well as other materials that may be the polymers of various embodiments of the invention, may include base resins that have been approved under one or both of USP Class VI standards and ISO 10993-1 standards. The United States Pharmacopeia (USP) is a private, non-governmental organization that promotes the public health by establishing state-of-the-art standards to ensure the quality of medicines and other health care technologies. Those standards include in vivo animal biological reactivity tests for elastomerics, plastics, and other polymeric material with direct or indirect patient contact. Plastics are categorized into six classes based on responses to a series of in vivo tests for which extracts, materials, and routes of administration are specified. Class VI requires the most stringent testing of the six classes. Class VI outlines requirements for system toxicity and intracutaneous toxicity for compounds. The USP Class VI compounds must be made from ingredients with clear histories of biocompatibility that meet tighter requirements for leachates. However, USP Class VI testing does not fully meet any category of ISO 10993-1 testing guidelines currently used by the US FDA for medical device approval.

The most influential guideline for biocompatibility is the ISO 10993-1 standard. This standard was developed for medical device and dental materials by the International Organization for Standardization. ISO 10993-1 provides for evaluation and testing of medical devices within a risk management process. The standard looks at the general classification of devices based on their nature and duration of contact with the body. It also explains the evaluation of existing and relevant data from all sources, before identifying information gaps. Best practice recommendations for accomplishing a full assessment of the biological safety of medical instruments and implants are also provided. ISO 10993-1 requires evaluation and testing of non-biological attributes and properties of materials used in medical devices, such as chemical, physical, electrical, morphological and mechanical properties.

Examples of suitable fillers include materials known for combination with polymers generally and polycarbonate specifically, as well as other materials as disclosed herein. Fillers may be for reinforcing a composition or for otherwise changing the physical or volumetric characteristics of a composition. Suitable fillers may include, without limitation, any reinforcing material or fiber. Specific reinforcing fillers include, but are not limited to, carbon fibers, short glass, glass fibers, graphite fibers, polymeric fibers, and aromatic polyimide (aramid) fibers. Fillers may include continuous or chopped fibers such as asbestos, carbon, or glass of any type or shape. Fillers may be elongated fibers with lengths five or more times greater than their widths.

Fillers may also include, without limitation, silicates and silica powders such as aluminum silicate (mullite), synthetic calcium silicate, zirconium silicate, fused silica, crystalline silica graphite, natural silica sand, boron powders such as boron-nitride powder, boron-silicate powders, oxides such as aluminum oxide, zirconium oxide, titanium dioxide, nanoscale titanium oxide, titanium boride, aluminum trihydrate, vanadium oxide, magnesium oxide, calcium sulfate, calcium carbonates such as chalk, limestone, marble, synthetic precipitated calcium carbonates, and talc, including fibrous, modular, needle shaped, lamellar talc. Fillers may include glass spheres such as hollow and solid glass spheres, silicate spheres, cenospheres, aluminosilicate (armospheres), kaolin, including hard kaolin, soft kaolin, calcined kaolin, kaolin comprising various coatings known in the art to facilitate compatibility with the polymeric matrix resin, single crystal fibers, silicon carbide, alumina, boron carbide, iron, nickel, copper, sulfides such as molybdenum sulfide, zinc sulfide, and barium compounds such as barium titanate, barium ferrite, barium sulfate, heavy spar. Fillers may also include metals and metal oxides such as particulate or fibrous aluminum, bronze, zinc, copper and nickel; flaked fillers such as glass flakes, flaked silicon carbide, aluminum diboride, aluminum flakes, steel flakes; fibrous fillers, and inorganic fibers such as those derived from blends comprising at least one of aluminum silicates, aluminum oxides, magnesium oxides, and calcium sulfate hemihydrate; or natural fillers such as wood flour obtained by pulverizing wood, fibrous products such as cellulose, cotton, sisal, jute, starch, cork flour, lignin, ground nut shells, corn, and rice grain husks. Fillers may include basalt fibers, carbon nanofibers, carbon nanotubes, carbon buckyballs, ultra high molecular weight polyethylene fibers, melamine fibers, polyamide fibers, potassium titanate whiskers, and aluminum borate whiskers. Fillers may also include organic fillers such as polytetrafluoroethylene, organic fibrous fillers formed from organic polymers capable of forming fibers such as poly(ether ketone), polybenzoxazole, poly(phenylene sulfide), polyesters, polyethylene, aromatic polyamides, aromatic polyimides, polyetherimides, polytetrafluoroethylene, acrylic resins, poly (vinyl alcohol), as well as additional fillers such as mica, clay, nano-clay, feldspar, flue dust, finite, quartz, quartzite, perlite, tripoli, diatomaceous earth, carbon black, or combinations comprising at least one of the foregoing fillers. Fillers may be of natural or synthetic, mineral or non-mineral origin, provided that the fillers have sufficient thermal resistance to maintain solid physical structure at least at the processing temperature of the polymer with which it is combined. Fillers may also include antimony trioxide, diatomaceous earth, fuller earth, kieselguhr, slate flour, volcanic ash, wollastonite, zinc borate, tungsten carbide, ferrites, molybdenum disulfide, cristobalite, and combinations comprising at least one of any of the fillers listed herein.

Suitable fillers may be provided in the form of monofilament or multifilament fibers and may be used either alone or in combination with other types of fiber, through any matrix or fibril construction, or by other methods known to one skilled in the art of fiber manufacture. Woven or mixed fillers structures may include, for example, glass fiber-carbon fiber, carbon fiber-aramid fiber, and aromatic polyimide fiberglass fiber or the like. Fibrous fillers may be supplied, without limitations, in the forms including but not limited to, ravings, woven fibrous reinforcements, non-woven fibrous reinforcements such as continuous strand mat, chopped strand mat, tissues, papers and felts or any three-dimensional reinforcement, such as a braid.

Polymers and fillers of embodiments of the invention may be consolidated together into a mixture with desirable characteristics. Any functional method of combining polymers and fillers may be used. Example methods of consolidating or mixing include a melt kneading method and a pultrusion method. The melt kneading method is a method wherein, with a polymer resin in a molten state, a filler is kneaded into the resin by an extruder. Such a melt kneading method may include one method (a side feeding method) wherein the resin is melted by a twin extruder, and the filler is introduced from a feed inlet. Another melt kneading method is a method (a premix method) wherein a resin and a filler that are preliminarily blended by a twin screw or single screw extruder, are melt-kneaded.

A pultrusion method also may be employed in a case where the form of the filler is a long fiber filler, and the molded product to be obtained is required to have high mechanical strength. The pultrusion method is one wherein while continuous long fiber strands are drawn, the resin to form a matrix is impregnated to the fiber strands. Pultrusion may also include a method wherein fiber strands are passed through an impregnation bath containing a solution of the matrix resin to impregnate the resin, a method wherein a powder of the matrix resin is sprayed on fiber strands, or fiber strands are passed through a tank containing the powder to attach the matrix resin powder to the fiber strands, and then, the matrix resin is melted and impregnated into the fiber strands, and a method wherein, while fiber strands are passed through a crosshead, the matrix resin is supplied to the crosshead to have the resin impregnated into the fiber strands. A polycarbonate resin composition produced by one of these methods may be subjected to a conventional molding method, such as injection molding, extrusion molding, compression molding, or calendaring to obtain a molded product. Further, the molding may be carried out by means of a mold having the interior covered with a resin film or a resin sheet.

Conditions for production of these types of composite materials, and the condition for molding the composite materials into a molded product can suitably be selected and are not particularly limited. However, a heating temperature during melt kneading or pultrusion processes or a temperature of the resin during injection molding may be within a range of from 220° C. to 300° C. in order to avoid decomposition of the resin.

The amount of filler used relative to the amount of polymer used in a mixture may affect the rigidity of the mixture when the mixture is cured. References to use of filler material herein include use of a single filler material and combinations of filler materials selected from the fillers specified. In some embodiments where the filler material comprises from 5 to 60 mass percent of the total mixture of polymer and filler, significant rigidity improvements to the cured mixture are accomplished without significantly reducing the material toughness of the cured mixture. In one such embodiment, the filler is short glass and the polymer is LEXAN HPH4404. Other polymers and fillers in similar proportions are contemplated to have similar properties. Another embodiment with effective cured material characteristics is a mixture of polymer with a filler material that is approximately 10 mass percent of the total mixture of polymer and filler. Another embodiment with effective cured material characteristics is a mixture of polymer with a filler material that is approximately 20 mass percent of the total mixture of polymer and filler. Another embodiment with effective cured material characteristics is a mixture of polymer with a filler material that is approximately 30 mass percent of the total mixture of polymer and filler. Another embodiment with effective cured material characteristics is a mixture of polymer with a filler material that is approximately 40 mass percent of the total mixture of polymer and filler. Another embodiment with effective cured material characteristics is a mixture of polymer with a filler material that is approximately 50 mass percent of the total mixture of polymer and filler.

An embodiment of the invention is a method of constructing a thermoplastic composite material that has the ability to be formed into a surgical instrument that is configured to receive an impact force. This method embodiment includes obtaining a polymer, such as any of the polymers referenced herein, including possibly a medical grade polymer. In some embodiments, the polymer may be a Poly (bisphenol-A-carbonate), and may be LEXAN HPH4404 particularly. The method also includes obtaining a filler material capable of increasing the rigidity of the thermoplastic composite material when consolidated with the polymer. A suitable filler material may be selected from the fillers referenced herein. In some embodiments, the filler may include a short glass material.

In an additional act of this embodiment, the polymer and the filler are consolidated to create a mixture. This consolidation may include a melt kneading method, a pultrusion method, or any other functional method of consolidating the materials to create a mixture. In a further act of the method the mixture may be pressed into a mold constructed in the shape of the surgical instrument that is configured to receive an impact force. Pressing of the mixture into the mold may be accomplished by the use of pneumatic, hydraulic, direct, or any sufficient force. Molds of various embodiments of the invention may be made in the shape of practically any desired implant or instrument shape and size. As an example, a mold may be constructed in the shape of the femoral trial 1 illustrated in FIGS. 1-2. As illustrated in FIG. 2, the femoral trial 1 includes an impaction zone 5 configured to receive an impact force. The femoral trial 1 is configured to receive an impact force at least because of the material toughness of the polymer from which it is partially formed.

Still other embodiments of the invention include a method of supplying a material acceptable for use as a surgical device. Such a surgical device may include instrument or implant devices. Such a method may include providing a polymer cleared for use during a surgical procedure for the benefit of a patient. A polymer cleared for use may include one or more of the following: approval from the US FDA, approval in Europe with a CE Mark, approval from a private standards body, approval from any accepted governmental or nongovernmental agency authorized to provide clearance in a particular jurisdiction, and approval in light of a particular clearance protocol. As described in greater detail herein, a polymer cleared for use may include one or both of a USP Class VI approved base resin and an ISO 10993-1 approved base resin. In some embodiments, the polymer may be a Poly (bisphenol-A-carbonate), and may be LEXAN HPH4404 particularly.

Such a method may further include providing a filler material configured to increase the rigidity of the polymer when consolidated with the polymer. A suitable filler material may be selected from the fillers referenced herein. In some embodiments, the filler may include a short glass material. The provided polymer and filler materials may be further consolidated to create a mixture in an additional act of the method embodiment. Consolidating the polymer with a filler to create a mixture may include any functional proportion of filler to polymer, including proportions specifically designated herein.

The method may also include evaluating the mixture for suitability. As used herein the term mixture includes characteristics of the mixture when cured. Such an evaluation may comprise conducting tests and analyses. Such an evaluation may also or in the alternative comprise providing information to another person, testing facility, or organization to conduct tests and analyses. For example, evaluating the mixture may include supplying one or both of samples and data to another person, testing facility, or organization that will use the samples or data to determine if the mixture is acceptable for use as a surgical device. Evaluation criteria may include inclusion of any desirable characteristic or exclusion of any undesirable characteristic, for example and without limitation, biocompatibility, material strength, durability, and rigidity.

In another act of some embodiments, the cured mixture comprising the final material is made available for use as a part of or as an entire surgical device. This act of making the material available includes making the material available for others to use to make surgical devices and includes using the material to make one's own devices, and then making those devices available for use. For example, the act of making the material available would include providing a material for the purchase of others who would manufacture a surgical device. The act of making the material available may also include manufacturing components, such as the femoral trial 1 illustrated in FIGS. 1-2, and making the femoral trial 1 available to medical providers.

Any embodiment or feature of any section, portion, or any other component shown or particularly described in relation to various embodiments of similar sections, portions, or components herein may be interchangeably applied to any other similar embodiment or feature shown or described herein. While embodiments of the invention have been illustrated and described in detail in the disclosure, the disclosure is to be considered as illustrative and not restrictive in character. All changes and modifications that come within the spirit of the invention are to be considered within the scope of the disclosure.

1. A femoral trial comprising:
a trial instrument made generally in the shape of a femoral component implant that is configured to receive impact to seat the trial instrument on a distal end of a femur of a patient;
wherein the trial instrument is manufactured at least in part from:
a medical grade polymer; and
a filler material capable of increasing the rigidity of the trial instrument when mixed with the medical grade polymer.

2. The formal trial of claim 1 wherein the trial instrument is a ream-through femoral trial.

3. The formal trial of claim 1 wherein the femoral trial is configured to be disposed of after a single use.

4. The formal trial of claim 1 wherein the medical grade polymer is a Poly (bisphenol-A-carbonate).

5. The formal trial of claim 4 wherein the Poly (bisphenol-A-carbonate) is LEXAN HPH4404.

6. The formal trial of claim 1 wherein the medical grade polymer includes one or both of a USP Class VI approved base resin and an ISO 10993-1 approved base resin.

7. The formal trial of claim 1 wherein the filler material is a reinforcing material.

8. The formal trial of claim 1 wherein the filler material is short glass.

9. The formal trial of claim 1 wherein the filler material includes elongated fibers.

10. The formal trial of claim 1 wherein the filler material comprises from 5 to 60 mass percent of the femoral trial.

11. The formal trial of claim 1 wherein the filler material is approximately 10 mass percent of the femoral trial.

12. The formal trial of claim 1 wherein the filler material is approximately 30 mass percent of the femoral trial.

13. The formal trial of claim 1 wherein the filler material is approximately 50 mass percent of the femoral trial.

14. A surgical instrument configured to be sterilized and used to manipulate the tissue of a patient comprising:
a surgical instrument body including a force receiving zone that is configured to receive impact to position the surgical instrument within the patient or to manipulate the tissue of the patient;
wherein the surgical instrument is manufactured at least in part from:
a medical grade polymer; and
a filler material capable of increasing the rigidity of the surgical instrument when mixed with the medical grade polymer.

15. The surgical instrument of claim 14 wherein the surgical instrument is configured to be disposed of after a single use.

16. The surgical instrument of claim 14 wherein the medical grade polymer is a Poly (bisphenol-A-carbonate).

17. The surgical instrument of claim 16 wherein the Poly (bisphenol-A-carbonate) is LEXAN HPH4404.

18. The surgical instrument of claim 14 wherein the medical grade polymer includes one or both of a USP Class VI approved base resin and an ISO 10993-1 approved base resin.

19. The surgical instrument of claim 14 wherein the filler material is a reinforcing material.

20. The surgical instrument of claim 14 wherein the filler material is short glass.

21. The surgical instrument of claim 14 wherein the filler material includes elongated fibers.

22. The surgical instrument of claim 14 wherein the filler material comprises from 5 to 60 mass percent of the surgical instrument.

23. The surgical instrument of claim 14 wherein the filler material is approximately 10 mass percent of the surgical instrument.

24. The surgical instrument of claim 14 wherein the filler material is approximately 30 mass percent of the surgical instrument.

25. The surgical instrument of claim 14 wherein the filler material is approximately 50 mass percent of the surgical instrument.

26. The surgical instrument of claim 14 wherein the surgical instrument is an instrument for use in an orthopedic procedure.

27. A method of constructing a thermoplastic composite material that has the ability to be formed into a surgical instrument that is configured to receive an impact force, the method comprising:
obtaining a medical grade polymer;
obtaining a filler material capable of increasing the rigidity of the thermoplastic composite material when consolidated with the medical grade polymer;
consolidating the medical grade polymer with the filler to create a mixture; and
pressing the mixture into a mold constructed in the shape of the surgical instrument configured to receive an impact force.

28.-38. (canceled)

39. A method of supplying a material acceptable for use as a surgical device comprising:
providing a polymer cleared for use during a surgical procedure for the benefit of a patient;
providing a filler material configured to increase the rigidity of the polymer when consolidated with the polymer;
consolidating the polymer with the filler to create a mixture;
evaluating the mixture for suitability; and
making the material available for use as part of a surgical device.

40.-55. (canceled)
